(19) United States
(12) Patent Application Publication
(10) Pub. No.: US 2020/0261618 A1
(43) Pub. Date: Aug. 20, 2020

(54) LIQUID TISSUE GRAFT

(71) Applicant: THE RESEARCH FOUNDATION FOR THE STATE UNIVERSITY OF NEW YORK, Syracuse, NY (US)

(72) Inventors: Dmitriy NIKOLAVSKY, Jamesville, NY (US); Gennady BRATSLAVSKY, Fayetteville, NY (US)

(21) Appl. No.: 16/067,875

(22) PCT Filed: Jan. 6, 2017
(86) PCT No.: PCT/US17/12569

§ 371 Date: Jul. 3, 2018

Related U.S. Application Data
(60) Provisional application No. 62/275,747, filed on Jan. 6, 2016.

(51) Int. Cl. A61L 27/36; A61L 27/54

(52) U.S. Cl. CPC A61L 27/3629; A61B 17/322; A61L 27/54; A61L 27/3687

(57) ABSTRACT

The present disclosure relates to methods and compositions comprising dissociated buccal mucosa tissue that are useful for treating a wound. Kits for preparing the liquid suspension of dissociated buccal mucosa tissue and treating the wound are also disclosed.

This application claims the benefit of U.S. Provisional Patent Application Ser. No. 62/275,747, filed Jan. 6, 2016, which is hereby incorporated by reference in its entirety.

This invention was made with government support under grant numbers EY019517 and EY017964 awarded by the National Institutes of Health. The government has certain rights in this invention.

### FIELD OF THE INVENTION

The present disclosure relates to methods for treating a wound that involve administering a liquid suspension of buccal mucosa tissue. Kits for preparing the liquid suspension of buccal mucosa tissue and treating the wound are also disclosed.

### BACKGROUND OF THE INVENTION

Urogenital stricture disease, which involves the abnormal narrowing of a ureter or urethra by inflammation or scar tissue, is a relatively common condition with no single optimal treatment modality. There are three options for treating strictured or scarred urogenital organs. The first option is dilation where the stricture is stretched using coaxial metal or plastic dilators (of increasing size) or a balloon catheter. This stretching is not a cure and needs to be periodically repeated. The second treatment option is the minimally invasive endoscopic direct vision internal urethrotomy (DVIU). In this procedure, a knife blade or laser at the end of a cytoscope is used to incise (cut through) the stricture to widen the urethral lumen in hopes of stabilizing the scar or re-epithelizing in open configuration. A stent or catheter is temporarily placed within the ureter or urethra to keep the passageway open and allow the surgical wound to heal. While the first two options are minimally invasive, they are also minimally effective with cure rates of 0-12%. The third treatment option is open reconstruction, a urethroplasty, which is a type of an invasive open procedure involving longer recovery. In this procedure, the strictured segment is either completely resected or longitudinally incised and the urethra or ureter is reconstructed by primary anastomosis or augmented/substituted using a free graft, skin flap, or intestinal segment (all depending on the location and length of stricture). While the open reconstruction is effective, it is known to be underutilized because specialized training and expertise are needed to perform the procedure and patients are reluctant to undergo such an invasive procedure. Accordingly, a minimally invasive, yet effective treatment option for stricture disease is warranted.

The present invention is directed to overcoming these and other deficiencies in the art.

### SUMMARY OF THE INVENTION

A first aspect of the disclosure is directed to a method of treating a wound in a subject. This method involves providing a liquid suspension of buccal mucosa tissue and dispersing the liquid suspension of buccal mucosa tissue over the wound thereby treating the wound in said subject. The wound can be an internal or external wound. An internal wound can be located within the urogenital tract duct or organ, the gastrointestinal tract duct or organ, or another lumen containing duct or organ of the subject.

Another aspect of the disclosure is directed to a method of treating a luminal stricture in a subject. This method involves removing or incising scar tissue associated with the luminal stricture, whereby said removing or incising causes a luminal wall defect. The method further involves providing a liquid suspension of buccal mucosa tissue and dispersing the liquid suspension of buccal mucosa tissue to cover the luminal wall defect, thereby treating the luminal stricture in the subject. The method may optionally include positioning a catheter, stent, inflatable balloon, or wound healing dressing within the lumen adjacent the luminal wall defect prior to dispersing the liquid suspension of buccal mucosa tissue to cover the luminal wall defect.

Another aspect of the present disclosure is directed to a method for preparing a liquid suspension of buccal mucosa tissue for use in the methods of treating a wound or a luminal stricture in a subject as described herein. This method involves harvesting buccal mucosa tissue from the subject, dissociating the harvested buccal mucosa tissue by mechanical, enzymatic or other means, and suspending the dissociated buccal mucosa in a carrier solution to form a liquid suspension of buccal mucosa tissue suitable for dispersing over a wound or luminal wall defect. The method may optionally include treating the buccal mucosa tissue prior to, during or after dissociation, such as treating the tissue with an antibiotic or a growth factor, adding one or more components to the liquid suspension that increase its viscosity, and/or filtering, purifying or concentrating the dissociated mucosa tissue. The method may optionally include expansion of cells in the buccal mucosa tissue prior to suspending the dissociated buccal mucosa in a carrier solution to form a liquid suspension. The liquid suspension of buccal mucosa tissue may contain epithelial progenitor cells from buccal mucosa tissue, other cells from buccal mucosa tissue, aggregates of cells from buccal mucosa tissue, and fragments of buccal mucosa tissue.

Another aspect of the present disclosure is directed to a liquid graft containing dissociated buccal mucosa tissue suspended in solution. The liquid graft may include epithelial progenitor cells, fragments of stratified squamous epithelium, and/or fragments of lamina propria. Another aspect of the present disclosure is directed to dissociated buccal mucosa tissue suspended in a solution that either contains one or more components that increase the viscosity of the solution or is viscous. The liquid graft may optionally contain one or more growth factors, one or more wound-healing factors, cell nutrient solution, saline solution, and/or antibiotic solution.

Another aspect of the present disclosure is directed to kit for preparing and/or delivering a liquid buccal mucosal tissue graft. This kit comprises instruments for dissociating buccal mucosa tissue, and one or more components suitable for suspension and/or delivery of the dissociated buccal mucosa tissue. The kit optionally contains instruments for harvesting buccal mucosa tissue from a patient; containers for housing harvested tissue; instruments and/or reagents for dissociating buccal mucosa tissue, such as an enzyme, chopper, sieve, mortar and pestle, scalpel and cutting surface, press mincer, and/or tissue grinder; instruments for containing the liquid buccal mucosal tissue graft at the intended site, such as a catheter, stent, inflatable balloon or wound healing dressing; an instrument for delivering and/or dispersing the liquid buccal mucosal tissue graft at the intended site, such a single, double, or triple barrel syringe; a viscous material or components capable of increasing the viscosity of a solution; and components to aid in the delivery and/or efficacy of the liquid buccal mucosal tissue graft, such as growth factors, wound-healing factors, cell nutrient solution, saline solution or antibiotic solution.

Another aspect of the present disclosure is directed to kit for preparing a liquid buccal mucosal tissue graft from disassociated buccal mucosa tissue or expanded cells from buccal mucosa tissue and delivering the liquid buccal mucosal tissue graft to an appropriate wound or surgical site. The kit comprises a viscous solution or components capable of increasing the viscosity of a solution and one or more instruments suitable for delivery of the liquid buccal mucosal tissue graft. The kit optionally comprises instruments for containing the liquid buccal mucosal tissue graft at the intended site, such as a catheter, stent, inflatable balloon or wound healing dressing; an instrument for delivering and/or dispersing the liquid buccal mucosal tissue graft at the intended site, such a single, double, or triple barrel syringe; and components to aid in the delivery and/or efficacy of the liquid buccal mucosal tissue graft, such as growth factors, wound-healing factors, cell nutrient solution, saline solution or antibiotic solution.

Described herein are methods of treating strictures and other wounds using a liquid suspension of dissociated buccal mucosa tissue. Sheaths of buccal mucosa are currently used in urethral reconstruction; however, urethral reconstruction is an invasive procedure that is infrequently employed. As described herein, an effective, yet non-invasive alternative treatment option involves the endoscopic delivery and application of an autologous liquid suspension of dissociated buccal mucosa tissue over a urethral defect created by removal of a stricture using DVIU. The liquid grafts disclosed herein can be prepared in minutes in the operating room concurrent with the surgical procedure. The method does not require a clinical worker to separate out from the buccal mucosa tissue specific cell types or separately culture and expand certain cells prior to their use for treating the patient, although expansion is an option. Upon delivery, the buccal mucosa tissue engrafts, survives, and cures the stricture. This technology is far less invasive than open reconstruction resulting in a shorter recovery time with a high cure rate. Overall, the technology is much less expensive than open reconstruction and easier for the general urologist to employ making it desirable for widespread adaption.

### BRIEF DESCRIPTION OF THE DRAWINGS

FIG. 1A is a photomicrograph of normal buccal mucosa (arrow) taken from the cheek of a rabbit stained with hematoxylin and eosin (H+E staining). FIG. 1B is a photomicrograph of a cross-section of normal untreated rabbit urethra with H&E staining. FIG. 1C shows a cross-section of rabbit urethra two weeks after liquid buccal mucosa administration (H&E staining). FIG. 1D shows a cross-section of rabbit urethra three weeks after liquid buccal mucosa administration (H&E staining).

FIGS. 2A-2D show radiological and endoscopic evidence of stricture formation in rabbits. FIGS. 2A, 2B, and 2C are retrograde urethrograms taken before (FIG. 2A), immediately after (FIG. 2B), and six weeks after (FIG. 2C) endoscopic stricture induction. FIG. 2D is an endoscopic view of strictured urethral segment.

FIG. 3A is a retrograde urethrogram of a stricture segment in a rabbit showing buccal mucosa tissue engraftment two weeks after treatment. FIG. 3B is a photomicrograph of the treated urethral segment two weeks post buccal mucosal tissue treatment.

FIGS. 4A-4C show stricture formation and treatment in a control animal. FIGS. 4A and 4B are retrograde urethrograms showing stricture before DVIU (FIG. 4A) and 8 weeks after DVIU without buccal mucosal graft (FIG. 4B). FIG. 4C is a photomicrograph of the urethra at the level of stricture 8 weeks post DVIU showing extensive collagen deposition and fibrosis (H&E staining).

FIGS. 5A-5C show stricture formation and treatment with the liquid buccal mucosa tissue graft. FIGS. 5A and 5B are retrograde urethrograms showing stricture before DVIU (FIG. 5A) and 8 weeks after DVIU with injection of buccal mucosal graft (FIG. 5B). FIG. 5C is a photomicrograph of the urethra at the level of stricture at 8 weeks after DVIU showing buccal mucosal engraftment within the urethra (H&E staining).

FIGS. 6A-6F show stricture formation and treatment with the liquid buccal mucosa tissue graft. FIGS. 6A and 6B are retrograde urethrograms showing stricture before DVIU (FIG. 6A) and 16 weeks after DVIU with injection of buccal mucosal graft (FIG. 6B). Corresponding retrograde urethrograms of the control animal, not receiving buccal mucosal engraftment are shown in FIGS. 6D and 6E, respectively. FIGS. 6C and 6F are photomicrographs of the urethra at the level of stricture at 16 weeks after DVIU. FIG. 6C shows buccal mucosal engraftment within the urethra (H&E staining), while FIG. 6F shows urethra of control animal that did not receive the buccal mucosal engraftment.

FIGS. 7A-7F show stricture formation and treatment with the liquid buccal mucosa tissue graft. FIGS. 7A and 7B are retrograde urethrograms showing stricture before DVIU (FIG. 7A) and 24 weeks after DVIU with injection of buccal mucosal graft (FIG. 7B). Corresponding retrograde urethrograms of the control animal, not receiving buccal mucosal engraftment are shown in FIGS. 7D and 7E, respectively. FIGS. 7C and 7F are photomicrographs of the urethra at the level of stricture at 24 weeks after DVIU. FIG. 7C shows buccal mucosal engraftment within the urethra (H&E staining), while FIG. 7F shows urethra of control animal that did not receive the buccal mucosal engraftment

### DETAILED DESCRIPTION OF THE INVENTION

The present disclosure relates generally to a dissociated buccal mucosa tissue graft and its utility for treating wounds. The disclosure also relates to kits for preparing the dissociated buccal mucosa tissue graft and wound healing compositions comprising the dissociated buccal mucosa tissue graft.

A first aspect of the disclosure is directed to a method of treating a wound in a subject. This method involves providing a liquid suspension of dissociated buccal mucosa tissue and dispersing the liquid suspension of dissociated buccal mucosa tissue over the wound thereby treating the wound in said subject.

In accordance with this and all aspects of the present disclosure, the liquid suspension of buccal mucosa tissue is a suspension of dissociated buccal mucosa tissue that comprises any one or more of the cell types present in the buccal mucosa. Buccal mucosa is a type of oral mucosa, known as lining mucosa. Oral mucosa is the mucous membrane lining the inside of the mouth and consists of stratified squamous epithelium termed oral epithelium and an underlying connective tissue termed lamina propria (the term mucosa or mucous membrane refers to the combination of the epithelium plus the lamina propria which line various tubes in the body, such as the respiratory tract, the gastrointestinal tract, and the urogenital tract). There are three categories of oral mucosa differentiated by their function and histology: masticatory mucosa, lining mucosa, and specialized mucosa. The lining mucosa is nonkeratinized stratified squamous epithelium covering the soft palate, inner lips, inner cheeks, and the floor of the mouth, and ventral surface of the tongue. In accordance with the present disclosure, buccal mucosa refers to the inside lining of the cheeks and is part of the lining mucosa.

The buccal mucosa consists of two layers, the surface stratified squamous epithelium and the deeper lamina propria. A stratified squamous epithelium consists of squamous (flattened) epithelial cells arranged in layers upon a basal membrane. Only one layer is in contact with the basement membrane (basal lamina), the interface between the oral epithelium and lamina propria; the other layers adhere to one another to maintain structural integrity. There are no intercellular spaces. Although this epithelium is referred to as squamous, many cells within the layers may not be flattened; in the deeper layers, the cells may be columnar or cuboidal. In addition to epithelial cells and epithelial progenitor cells, other cell types present in the squamous epithelium layer of the buccal mucosa are Merkel cells, melanocytes, lymphocytes, Langerhans cells and neutrophils. The liquid suspension of buccal mucosa used in the methods described herein may comprise any one or more of these cell types. In one embodiment, the liquid suspension of buccal mucosa comprises epithelial cells, epithelial progenitor cells, or a mixture of both. In another embodiment, the liquid suspension of buccal mucosa further comprises Merkel cells, melanocytes, lymphocytes, Langerhans cells and/or neutrophils. The liquid suspension of buccal mucosa may comprise expanded populations of any of the aforementioned cell types, where said expanded populations are generated via ex vivo culturing of the buccal mucosa.

The lamina propria or lamina propria mucosa is a thin layer of loose connective tissue, or dense irregular connective tissue, which lies beneath the epithelium. The interface between the epithelium and the lamina propria can be irregular, with portions of each layer projecting into the other, sometimes considerably. The resulting interdigitation increases the area of contact between the two layers. The lamina propria consists of a network of type I and type III collagen and elastin fibers in some regions. While the main cells of the lamina propria are the fibroblasts, which are responsible for the production of the fibers as well as the extracellular matrix, also present are lymphocytes, plasma cells, macrophages, eosinophilic leukocytes, and mast cells. The liquid suspension of buccal mucosa used in the method described herein may further comprise any one or more of these cell types. In one embodiment the liquid suspension of buccal mucosa tissue comprises fibroblasts and epithelial cells, i.e., epithelial progenitor cells and/or epithelial cells. In another embodiment, the liquid suspension of buccal mucosa tissue further comprises lymphocytes, plasma cells, macrophages, eosinophilic leukocytes, and mast cells. The liquid suspension of buccal mucosa may comprise expanded populations of any of the aforementioned cell types, where said expanded populations are generated via ex vivo culturing of the buccal mucosa. In another embodiment, the liquid suspension of buccal mucosa tissue comprises epithelial progenitor cells along with collagen, e.g., type I or III collagen, and/or elastin.

The buccal mucosa can be harvested using standard procedures well known to those of skill in the art, e.g., incisional biopsy, excisional biopsy, punch biopsy, and brush biopsy. The size of the buccal mucosa biopsy required for preparation of the liquid suspension of buccal mucosa tissue will vary depending on the size of the wound or defect to be treated, typically ranging between 4 mm in diameter to 11×2 cm (per cheek). In one embodiment, the biopsy is collected from the subject having the wound or having a procedure requiring a liquid graft, such as treatment of a urethral stricture, i.e., the suspension of liquid buccal mucosa tissue is produced from an autologous tissue biopsy. In another embodiment, the biopsy is collected from a donor subject other than the subject having the wound, i.e., the suspension of liquid buccal mucosa tissue is produced from a non-autologous tissue biopsy. In either embodiment, the biopsied buccal mucosa tissue is optionally rinsed in an antibiotic after harvesting.

To prepare a liquid suspension of buccal mucosa tissue for a liquid graft as herein described, the buccal mucosa biopsy is dissociated, i.e., broken up into smaller pieces or fragments. The buccal mucosa tissue can be dissociated mechanically, chemically, enzymatically, or using a combination of these techniques. After dissociation, the dissociated buccal mucosa tissue may include lysed cells and their components, buccal mucosa extracellular matrix, buccal mucosa interstitial fluid (and all it contains), plasma (and all it contains), single cells, and aggregates of cells. Aggregates of cells include those formed from dissociated cells which aggregate after dissociation as well as aggregates of cells in fragments of the original buccal mucosa tissue biopsy.

As used here, a fragment of buccal mucosa tissue is a piece or segment of the original buccal mucosa tissue biopsy that maintains some or all of its native structure; i.e., the cells in the fragment have not been fully dissociated from one another and are present in the fragment in fundamentally the same configuration relative to one another as they were in the original tissue. The dissociation process may cause alterations to the fragment's internal and external structure and organization. For example, a fragment may contain a piece of the stratified squamous epithelium (not necessarily the entire thickness) connected to piece of the lamina propria (not necessarily the entire thickness) connected, as in situ in the inner cheek, via the basal lamina.

The fragments of buccal mucosa tissue can comprise fragments of the stratified squamous epithelium, fragments of the lamina propria, and/or fragments containing at least part of both the stratified squamous epithelium and the lamina propria. Some fragments may not contain any cells, but only extracellular matrix and/or other non-cell components of the buccal mucosa (e.g., plasma, extracellular proteins, signaling molecules, RNAs, etc.), while other fragments may contain both cellular and non-cellular buccal mucosa components, and other fragments may contain only buccal mucosa cells. The fragments of buccal mucosa tissue can have volumes ranging in size from approximately 0.25 picoliter to tens of microliters, including, without limitation, volumes of 1 picoliter (10 microns cubed), 10 picoliters, 100 picoliters, 1 microliter (1 mm cubed), and 10 microliters. The suspended dissociated buccal mucosa tissue may contain cell aggregates of the following size ranges, inclusive: 0.25 picoliter to 50 picoliters; 0.25 picoliter to 100 picoliters; 0.25 picoliter to 250 picoliters; 0.25 picoliter to 500 picoliters; 0.25 picoliter to 1 microliter; 10 picoliters to 100 picoliters; 10 picoliters to 1 microliter; 0.25 picoliters to 10 microliters.

Aggregates of buccal mucosa cells can range from a small number cells (e.g., 2) to submillimeter- and millimeter-scale fragments of buccal mucosa tissue containing tens, hundreds, thousands, tens or hundreds of thousands, millions, tens or hundreds of millions, or billions of cells. Cell aggregates may constitute a single cell type or multiple cell types of the various cell types found in the buccal mucosa as well as biological matter, such as extracellular matrix, interstitial fluid, plasma or proteins, which are found in or are part of buccal mucosa tissue.

Methods of mechanical dissociation of the harvested buccal mucosa tissue include without limitation mincing, sieving, grinding, pipetting, titrating, homogenizing, shaking, sonicating, freeze-thawing, grating, scraping, and rolling the harvested tissue until the desired dissociation is achieved (i.e., dissociation of the tissue into single cells, cell aggregates, sub-millimeter, millimeter fragments, and any combination thereof as described above). In one embodiment, the harvested buccal mucosa tissue is dissociated to the extent necessary to allow for delivery of the liquid suspension via a catheter, syringe or other suitable delivery device. For example, if a syringe having a needle with 1 mm ID is used to deliver the liquid suspension of dissociated buccal mucosa tissue, the dissociated fragments must be small enough (e.g., less than about 1 mm) to pass easily through the needle.

Enzymatic dissociation of the harvested buccal mucosa tissue can be achieved by incubating the harvested tissue with one or more tissue-dissociating enzymes such as, for example, and without limitation trypsin, papain, elastase, pronase, hyaluronidase, collagenase, Dispase®, Liberase®, neutral protease, deoxyribonuclease I, or any combination of these enzymes until the desired dissociation is achieved (i.e., dissociation of the tissue into single cells, cell aggregates, sub-millimeter fragments, and any combination thereof.)

Depending on the means of dissociation employed, the liquid suspension of buccal mucosa tissue comprises at least one of buccal mucosa cells, buccal mucosa cell aggregates, and sub-micrometer to sub-millimeter to millimeter buccal mucosa fragments. In one embodiment, the liquid suspension comprises a mixture of buccal mucosa cells, cell aggregates and/or buccal mucosal fragments. Optionally, and depending on the wound to be treated, the suspension of liquid buccal mucosa tissue may be subjected to one or more separation, purification, or enrichment steps following dissociation for size selection or cell selection purposes. Such purification techniques are readily known in the art, including separation by cell surface protein expression (e.g., FACS sorting, magnetic cell sorting, adhesion based sorting, complement depletion), density centrifugation, and/or filtration (see generally, Tomlinson et al., “Cell Separation: Terminology and Practical Considerations,” J. Tissue Engineer. 4: 1 (2013), and Amos et al., “Methods of Cell Purification: A Critical Juncture for Laboratory Research and Translational Science,” Cells Tissues Organs 195(1-2): 26-40 (2012), which are hereby incorporated by reference in their entirety). In one embodiment, epithelial progenitor cells in the dissociated buccal mucosa tissue are separated out of the suspension or are concentrated such that the liquid suspension contains only epithelial progenitor cells or contains a higher concentration of epithelial progenitor cells than normal buccal mucosa tissue, e.g., epithelial progenitor cells may comprise about 20%, 30%, 40%, 50%, 60%, 70%, 80%, or 90% of the total cells in the suspended tissue. In one embodiment, fragments of dissociated buccal mucosa tissue with sizes or volumes at or above about 10 picoliters, or about 100 picoliters, or about 250 picoliters, or about 500 picoliters, or about 1 microliter, or about 2, 3, 5, or 10 microliters are removed through a filtration or separation process.

In one embodiment, the buccal mucosa biopsy is obtained and the liquid suspension of buccal mucosa tissue is prepared for immediate use in wound treatment. In another embodiment, a buccal mucosa tissue biopsy is obtained and frozen, and then the liquid suspension of buccal mucosa tissue is prepared using the frozen biopsy for use in wound treatment. In another embodiment, the liquid suspension of buccal mucosa tissue is obtained and frozen and then thawed prior to use in wound treatment. To increase the amount of buccal mucosa tissue available for the liquid graft, multiple biopsies can be collected at different times from the same subject, each biopsy being frozen at the time of collection for preservation, before or after suspension, and later thawed and combined prior to wound treatment. In another embodiment, the biopsied buccal mucosa tissue is, or selected buccal mucosa cells are, cultured to increase the amount of tissue or number of cells available for grafting (such as, for example, where the wound to be treated is large). Any of the steps or processes described herein as being done to a buccal mucosa biopsy can be done to cultured buccal mucosa tissue or cells.

Following dissociation, the dissociated buccal mucosa tissue is suspended in one or more solutions for washing, storage (temporary or long-term storage solution), and/or delivery (i.e., a carrier solution). In some embodiments, these solutions are the same solution. In some embodiments, these solutions comprise one or more different solutions depending on, among other things, the size, type, and location of the wound to be treated.

The washing, storage and/or delivery solutions optionally contain one or more of a pharmaceutically acceptable saline solution (e.g., physiological or sterile saline solution), phosphate buffered saline (PBS), Ringer's serum, Ringer's lactate serum, autologous serum, and AB donor serum, and other excipients such as 4-(2-hydroxyethyl)-1-piperazine-ethanosulfonic acid (HEPES) buffer, bicarbonate buffer, heparins, platelet lysates, and N-acetyl cysteine (NAC) to maintain cell viability and integrity prior to, during, or after dispersion at the wound site.

The washing, storage, and/or delivery solutions may optionally also contain one or more growth factors, one or more antibiotics, one or more agents known to promote wound healing, or a combination thereof. Suitable growth factors include, without limitation, platelet derived growth factor (PDGF), tumor necrosis factor-alpha (TNF-α), TNF-β, epidermal growth factor (EGF), keratinocyte growth factor, vascular endothelial growth factors (VEGFs), fibroblast growth factors (FGFs), tumor necrosis factor, insulin growth factor (IGF), and any combination thereof.

Suitable antibiotics that can optionally be included in the washing, storage, and/or delivery solutions include, for example and without limitation, aminoglycoside antibiotics (e.g., gentamycin, tobramycin), quinolones, beta-lactams (e.g., ampicillin, cephalosporins), ciprofloxacin, erythromycin, vancomycin, oxacillin, cloxacillin, methicillin, lincomycin, and colistin. Suitable antimicrobials include, for example, Adriamycin PFS/RDF® (Pharmacia and Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin® PFS (Pharmacia and Upjohn), Mithracin® (Bayer), Mitamycin® (Bristol-Myers Squibb Oncology/Immunology), Nipen® (SuperGen), Novantrone® (Immunex) and Rubex® (Bristol-Myers Squibb Oncology/Immunology). The antibiotics are administered in any suitable dosage that is effective to induce or to enhance the desired wound healing. Preferably, the lowest effective dose that contributes to the desired wound healing is utilized.

Suitable wound healing promoter agents that are optionally included in the liquid suspension of buccal mucosa tissue include without limitation extracellular matrix proteins such as glycosaminoglycan, proteoglycans, collagen, fibronectin, elastin, laminin, alginate, a chitin derivative, fibrin, fibrinogen, and any combination thereof. Other suitable wound-healing agents include, for example, vitamin A, aminoxyls, furoxans, nitrosothiols, nitrates and anthocyanins; nucleosides, such as adenosine; nucleotides, such as adenosine diphosphate (ADP) and adenosine triphosphate (ATP); histamine and catecholamines; lipid molecules, such as sphingosine-1-phosphate and lysophosphatidic acid; amino acids, such as arginine and lysine; peptides such as the bradykinins, substance P and calcium gene-related peptide (CGRP), and proteins, such as insulin, vascular endothelial growth factor (VEGF), and thrombin. The wound healing promoting agents are present in the carrier solution at a dosage that is effective to induce or to enhance the desired wound healing. Preferably, the lowest effective dose that contributes to the desired wound healing is utilized.

Delivering the dissociated buccal mucosa tissue to the site of a wound or luminal wall defect in a carrier solution that is viscous or becomes viscous once dispersed helps the liquid graft remain at the wound site. In some embodiments, the dissociated buccal mucosa tissue is suspended in a non viscous carrier solution that is made viscous by the addition of one or more reagents or by exposure to some outside factor (e.g., light, heat). In some embodiments the dissociated buccal mucosa tissue is mixed into an already viscous carrier solution, such as a gel or medical honey. In some embodiments, the dissociated buccal mucosa tissue is suspended in a non viscous carrier solution which is held at the wound site mechanically, such as by a porous material, a wound healing dressing or a stent coated with an absorbant material into or onto which the carrier solution has previously been dispersed or placed and which is then placed adjacent to the wound.

In some embodiments, the delivery solution (also referred to herein as the carrier solution) of the buccal mucosa tissue comprises one or more components that increase the viscosity of said suspension either prior to, concurrent with, or after dispersing the suspension over the wound to be treated. The choice of what component to use to increase the viscosity of the buccal mucosa tissue suspension is based on, among other factors, the type and location of the wound to be treated and means of dispersing and delivering the liquid buccal mucosa tissue.

In one embodiment, the component of the suspension that increases viscosity is a fibrin sealant. Fibrin sealants generally consist of two human plasma-derived components: (i) a highly concentrated Fibrinogen Complex (FC) composed primarily of fibrinogen and fibronectin along with catalytic amounts of Factor XIII and plasminogen, and (ii) a high potency thrombin. Fibrin sealants may also contain aprotinin. By the action of thrombin, (soluble) fibrinogen is first converted into fibrin monomers which aggregate spontaneously and form a fibrin clot. Simultaneously, factor XIII (FXIII) present in the solution is activated by thrombin in the presence of calcium ions to factor XIIIa. The aggregated fibrin monomers and any remaining fibronectin present are cross-linked to form a high molecular weight polymer by new peptide bonds forming. By this cross-linking reaction, the strength of the clot formed is substantially increased. Suitable fibrin sealants for use in the methods disclosed herein are known in the art, see e.g., U.S. Patent Publication No. 2008/0181879, which is hereby incorporated by reference in its entirety, and are commercially available, e.g., TISSEEL® (Baxter), BERIPLAST® (Behringwerke AG, Marburg/Lahn, FRG) and BIOCOL® (CRTS, Lille, France).

In one embodiment, the two components of the fibrin sealant, i.e., the fibrinogen complex (FC) component and the thrombin component are mixed with the liquid suspension of the buccal mucosa prior to dispersion. Alternatively, to prevent the liquid suspension of buccal mucosa from becoming viscous prior to delivery, the FC component and thrombin component are separated and combined at the time of delivery via simultaneous administration, or combined after delivery via consecutive delivery to the wound. In accordance with these embodiments, the liquid suspension of buccal mucosa is added to the solution containing the FC component or to the solution containing the thrombin component, is divided between the two, or is added after the FC-containing solution and the thrombin-containing solution are mixed. The FC and thrombin containing solutions are then dispersed on the wound simultaneously or consecutively so that the solution becomes viscous at the time of or after dispersion onto the wound. In some embodiments, depending on the wound being treated and the clotting factors present at the wound site, delivery of only one component will be sufficient to achieve the increase in viscosity of the carrier solution necessary to treat the wound. Other two component solutions, where mixing the two components results in a single viscous solution, can be used similarly to provide a carrier solution for the dissociated buccal mucosa tissue.

The FC/thrombin ratio may be between the range of 2 to 10 mg/ml fibrinogen per 25 U/ml thrombin, which includes, for example, about 2 mg/ml fibrinogen per about 25 U/ml thrombin, about 2.5 mg/ml fibrinogen per about 25 U/ml thrombin, about 3 mg/ml fibrinogen per about 25 U/ml thrombin, about 3.5 mg/ml fibrinogen per about 25 U/ml thrombin, about 4 mg/ml fibrinogen per about 25 U/ml thrombin, about 4.5 mg/ml fibrinogen per about 25 U/ml thrombin, about 5 mg/ml fibrinogen per about 25 U/ml thrombin, about 5.5 mg/ml fibrinogen per about 25 U/ml thrombin, about 6 mg/ml fibrinogen per about 25 U/ml thrombin, about 6.5 mg/ml fibrinogen per about 25 U/ml thrombin, 7 about mg/ml fibrinogen per about 25 U/ml thrombin, about 7.5 mg/ml fibrinogen per about 25 U/ml thrombin, about 8 mg/ml fibrinogen per about 25 U/ml thrombin, about 8.5 mg/ml fibrinogen per about 25 U/ml thrombin, about 9 mg/ml fibrinogen per about 25 U/ml thrombin, about 9.5 mg/ml fibrinogen per about 25 U/ml thrombin, and about 10 mg/ml fibrinogen per about 25 U/ml thrombin.

Thrombin is added to the fibrinogen solution in an amount sufficient to form a polymerized gel within between about 3 seconds to about 120 seconds. Preferably, between about 3 seconds to about 60 seconds, between about 3 seconds to about 30 seconds, between about 3 seconds to about 20 seconds, between about 3 seconds to about 10 seconds, between about 10 seconds to about 60 seconds, between about 20 seconds to about 60 seconds, between about 30 seconds to about 60 seconds, or between about 30 seconds to about 120 seconds.

In another embodiment, the suspension of buccal mucosa tissue is mixed with a natural, semi-synthetic, or synthetic polymer that polymerizes in situ to form a hydrogel. A number of biocompatible, biodegradable hydrogels that polymerize in response to chemical, thermal or optical triggers are known in the art and are suitable for use in the methods disclosed herein. For example, and without limitation, suitable hydrogel materials include alginate hydrogels (Bidarra et al., “Injectable Alginate Hydrogels for Cell Delivery in Tissue Engineering,” Acta Biomaterialia 10(4): 1646-62 (2014); Kong et al., “The Effects of Poly(ethyleneimine) (PEI) Molecular Weight on Reinforcement of Alginate Hydrogels,” Cell Transplant 12(7): 779-785 (2003); and Aguado et al., “Improving Viability of Stem Cells During Syringe Needle Flow Through the Design of Hydrogel Cell Carriers,” Tissue Engineering: Part A 18(7): 806 (2012), which are hereby incorporated by reference in their entirety), polyethylene glycol-based hydrogels (Bjugstad et al., “Biocompatibility of PEG-Based Hydrogels in Primate Brain,” Cell Transplant 17(4): 409-415 (2008), which is hereby incorporated by reference in its entirety), hyaluronic acid hydrogels (e.g., HyStem-C) (Zheng et al., “In situ Crosslinkable Hyaluronan Hydrogels for Tissue Engineering,” Biomaterials 25:1339-1348 (2004), which is hereby incorporated by reference in its entirety), hyaluronan and methylcellulose based hydrogels (Ballios et al., “A Hydrogen-based Stem Cell Delivery System to Treat Retinal Degenerative Diseases,” Biomaterials 31(9): 2555-64 (2010), which is hereby incorporated by reference in its entirety), protein-polyethylene glycol (PEG) hybrid hydrogels (Mulyasasmita et al., “Avidity-controlled Hydrogels for Injectable Co-delivery of Induced Pluripotent Stem Cell-derived Endothelial Cells and Growth Factors,” J. Control. Release 191: 71-81 (2014), which is hereby incorporated by reference in its entirety), collagen I hydrogels (e.g., Purecol®) (Beckman and Diegelman (eds) Collagen: Informa Healthcare USA (2008), which is hereby incorporated by reference in its entirety), and peptide polymer hydrogels such as Puramatrix™, composed of repeating amino acid sequences of Arginine-Alanine-Aspartic Acid-Alanine (Moon et al., “Micropatterning of Poly(ethylene glycol) Diacrylate Hydrogels with Biomolecules to Regulate and Guide Endothelial Morphogenesis,” Tissue Eng. Part A 15(3):579-585 (2009), which is hereby incorporated by reference in its entirety).

In one embodiment, the hydrogel component of the carrier solution of the liquid suspension of buccal mucosa tissue is a thermosensitive component that gelatinizes when the temperature reaches 37° C., e.g., when injected into the body. Both natural (e.g., collagen, gelatin, and chitosan) and synthetic polymers can be incorporated into thermosensitive solutions to produce a liquid suspension of buccal mucosa tissue that becomes viscous after administration or dispersion on a wound. Non-limiting examples of thermosensitive hydrogel materials known to be useful for encapsulating living cells for in situ deliver include mixtures of the polysaccharide chitosan and β-glycerophosphate (β-GP) (Chenite et al., “Novel Injectable Neutral Solutions of Chitosan Form Biodegradable Gels In situ,” Biomaterials 21(21): 2155-61 (2000), which is hereby incorporated by reference in its entirety), chitosan-Pluronic hydrogels (Park et al., “Thermosensitive Chitosan-Pluronic Hydrogel as an Injectable Cell Delivery Carrier for Cartilage Regeneration,” Acta Biomaterialia 5(6):1956-1965 (2009), which is hereby incorporated by reference in its entirety), co-polymers of N-isopropylacrylamide and acrylic acid poly(N-isopropylacrylamide-co-acrylic acid (Park and Yun, “Immobilization of Arg-Gly-Asp (RGD) Sequence in a Thermosensitive Hydrogel for Cell Delivery Using Pheochromocytoma Cells (P12),” J. Biosci. Bioengineer. 97(6):374-77 (2004), which is hereby incorporated by reference in its entirety), and poly(ethylene oxide)/poly(propylene oxide) block copolymers and poly(ethylene glycol)/poly(D,L-lactide-co-glycolide) block copolymers (Jeong et al., “Thermosensitive Sol-Gel Reversible Hydrogels,” Advanced Drug Delivery Reviews 64S:154-62 (2012), which is hereby incorporated by reference in its entirety).

In another embodiment, the hydrogel component of the carrier solution of the liquid suspension of buccal mucosa tissue contains a photosensitive component, i.e., a photoinitiator, which allows for spatial and temporal control of polymerization of the suspension by exposure to light. For example, in one embodiment the photoinitiator is the water-soluble two-photon active molecule, 1,4-bis(4-(N,N-bis(6-(N,N,N-trimethylammonium)hexyl)amino)-styryl)-2,5-dimethoxybenzene tetraiodide as described in Torgersen et al., “Photo-sensitive Hydrogels for Three-dimensional Laser Microfabrication in Presence of Whole Organisms,” J. Biomed. Optics 17(10): 105008 (2012), which is hereby incorporated by reference in its entirety. In another embodiment, the photoinitiator comprises IRGACURE® 2959 as described in Ovsianikov et al., “Laser Fabrication of Three-Dimensional CAD Scaffolds from Photosensitive Gelatin for Applications in Tissue Engineering,” Biomacromolecules 12(4):851-858 (2011), which is hereby incorporated by reference in its entirety. Other commonly employed photoinitiators used in preparing hydrogels include, without limitation, IRGACURE® 651, lithium phenyl-2,4,6-trimethylbenzoylphosphinates (LAP), Eosin Y, and cyclic benzylidene ketone-based photoinitiators (e.g., P2CK), see Qin et al., “Additive Manufacturing of Photosensitive Hydrogels for Tissue Engineering Applications,” BioNanoMaterials 15(3-4): 49-70 (2014), which is hereby incorporated by reference in its entirety.

In embodiments were the carrier solution comprises a photoinitiator component to control viscosity, the method of treating a wound as described herein further involves exposing the liquid suspension of buccal mucosa tissue to light of the appropriate wavelength to effectuate the change in viscosity. Exposing the liquid suspension of buccal mucosa tissue to light can occur before, during, or immediately after dispersion of the liquid suspension onto or into the site to be treated. When the exposing is carried out before administration of the liquid suspension, the amount of time between exposure and administration will be determined by the time it takes for the photosensitive component to induce a change in the viscosity of the suspension. Use of a photosensitive component may further necessitate shielding the liquid suspension from light, such as by administering it using a dispersal device that is opaque to the relevant wavelength.

In some embodiments, the suspension of buccal mucosa tissue is mixed into a viscous material. In one embodiment, the suspension of buccal mucosa tissue is mixed with medical honey to enhance viscosity for administration to a wound. Medical grade honey products such as MEDIHONEY® are commercially available and known to exhibit antibacterial properties and promote wound healing, making it a suitable delivery vehicle for the buccal mucosa tissue in the methods disclosed herein.

In another embodiment, the suspension of buccal mucosa tissue is mixed with a lubricating gel, such as a glycerin based gel (e.g., ELASTO-GEL™) or a bacitracin gel. If the dissociated buccal mucosa tissue is mixed into a gel or other viscous material prior to application to a wound, such suspension of dissociated buccal mucosa tissue in a viscous material may be applied to the wound through a tube or catheter of suitably large gauge attached to a syringe or bulb or using an applicator (for example, using a brush or roller to pick up the suspension of dissociated buccal mucosa tissue and then deposit it at the wound site in the same way that a paint brush or roller is used to pick up paint and deposit it on an item being painted), or applied first to a solid carrier (for example, a wound healing dressing or stent) which is then applied at or over the wound site with the suspension of dissociated buccal mucosa tissue in contact with the wound.

For the treatment of some wounds, e.g., external wounds, the dissociated buccal mucosa tissue, in suspension or not, may be embedded in a wound healing dressing to facilitate maintained dispersion of the suspension. The wound dressing material can be any material applied to a wound for protection, absorbance, drainage, etc. Numerous types of dressings are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), cellophane (cellulose with a plasticizer), gauze, alginate, polysaccharide paste, granules, and beads. The dissociated buccal mucosa tissue may also be embedded in or held on a stent that is placed at the wound site and serves to both deliver and disperse the liquid graft and hold it in place.

Suitable wounds to be treated in accordance with this aspect of the disclosure include any type of internal or external wound. In one embodiment, the wound is an acute wound. In another embodiment, the wound is a chronic wound. In one embodiment, the wound is a surgically induced wound. In another embodiment, the wound is a non-surgically related wound.

In one embodiment, the wound is an external wound, such as chronic or acute cutaneous lesions. Accordingly, in some embodiments, the liquid suspension of dissociated buccal mucosa tissue is administered topically to the surface of skin lesions, such as chronic ischemic skin lesions, chronic skin ulcers, and/or diabetic foot ulcers. Other types of skin lesions that can be treated using the methods described herein include neuropathic and ischemic chronic cutaneous lesions (e.g., low-grade lesions, grade IV lesions, grade V lesions, etc.), lacerations, and burn wounds.

In another embodiment, the wound to be treated is an internal wound. An “internal wound” as used herein refers to any wound or injury to an internal organ. Internal organs include, without limitation, organs of the gastrointestinal tract (e.g., esophagus, stomach, duodenum, small intestines, colon and recto-anal junction, anus), the urinary system (kidney, ureter, bladder and urethra), the reproductive system (testes, ductuli efferentes, epididymis, ductus deferens, ejaculatory duct, seminal vesicles, prostrate gland, penis, Cowper gland, ovary, oviduct, uterus and vagina), the immune system (spleen, thymus and lymph nodes), the respiratory system (nasal cavity, trachea, bronchi, bronchioli, alveoli and visceral pleura), the endocrine glands (pituitary, thyroid, parathyroid, adrenal, endocrine pancreas and pineal), the heart, heart valves, lymph vessels, liver, gall bladder, exo-pancreas, spleen, bone, tendon, muscle, intervertebral joints, salivary glands, olfactory receptors, eyes and ears.

In one embodiment, the internal wound is located within a lumen containing duct or organ of said subject, including, for example, and without limitation a duct or organ within the urogenital tract (e.g., ureter, urethra, bladder) or gastrointestinal tract (e.g., esophagus, intestines, anus). In one embodiment, the internal wound to be treated is a surgically induced wound, e.g., caused by removal, incision, or disruption of scar tissue within the lumen containing duct or organ of said subject.

One aspect of the disclosure is directed to a method of treating a luminal stricture in a subject. This method involves removing, incising or otherwise disrupting scar tissue associated with the luminal stricture, whereby said removing, incising or disrupting causes a luminal wall defect or wound. The method further involves providing a liquid suspension of buccal mucosa tissue and dispersing the liquid suspension of buccal mucosa tissue to cover the luminal wall defect, thereby treating the luminal stricture in the subject. The method of dispersing the liquid suspension of buccal mucosa tissue may include the step of using an endoscopic instrument for observation of the procedure (e.g., to be sure that the dispersion is done at the site of the wound) or for both observing the procedure and dispersing the liquid suspension of buccal mucosa tissue (e.g., through a lumen attached to the endoscopic instrument).

In one embodiment, the luminal stricture is a stricture found within a duct or tube of the urogenital tract, e.g., a urethral stricture, ureteral stricture, or bladder neck contracture. In another embodiment, the luminal stricture is a stricture found within a duct or tube of the gastrointestinal tract, e.g., esophageal stricture, intestinal strictures, anal stricture or fissure.

In accordance with this aspect of the disclosure, the scar tissue associated with the luminal stricture is removed, incised or otherwise disrupted endoscopically by cutting, incising, resecting, or scoring the scar tissue using a blade (or the like) or laser. Removal, incision or disruption of the scar tissue produces a luminal wall defect or wound that is treated by application of the liquid suspension of buccal mucosa tissue as described herein. The removal, incising or disruption of scar tissue may be accomplished with the aid of an endoscopic device, including an endoscopic device with an attachment or end adapted for incising, removing or otherwise disrupting tissue.

In one embodiment, a catheter or the like is positioned within the lumen adjacent the luminal wall defect or wound after the stricture tissue is removed, incised or otherwise disrupted, and the liquid suspension of buccal mucosa tissue is dispersed around the outside of the catheter, i.e., between the luminal wall defect and outside catheter wall. Use of the catheter provides a mold for the liquid suspension of dissociated buccal mucosa tissue and reduces the volume of the liquid suspension needed to cover the luminal wall defect. The catheter also serves to maintain the passageway of the lumen or duct containing the stricture. For example, when treating a urethral stricture, the catheter serves to maintain the passageway for urine flow out of the body and protects the luminal defect site from exposure to the passing urine. Inflatable instruments, such as those used in balloon angioplasty or urethroplasty, can be also used for a similar purpose, particularly when it will be kept in place for a short amount of time. A stent can also be used for similar purpose for wounds inside a lumen. For wounds on a larger surface, a wound healing dressing can be used to hold the liquid suspension of dissociated buccal mucosa tissue in place.

The catheter (or other device holding the liquid graft in place or protecting the wound) is removed some time after the dispersion of the liquid suspension of dissociated buccal mucosa tissue over the luminal defect, for example, the catheter (or other device holding the liquid graft in place or protecting the wound) is removed 1, 2, 3, 4, 5, 6, 7, 8, 9, 10, 11, 12, 13, or more days after the dispersion of the liquid buccal mucosa tissue over the luminal defect.

The proposed textbook mechanism for engraftment states that grafts receive oxygenation and nutrition via imbibition, which takes 36-48 hours and maintains cellular viability until the processes of inosculation and revascularization have re-established a new blood supply within the graft over the next 36-48 hours. Thus according to theory, in order to ensure full engraftment any instrument serving as a mold to hold the graft in place should stay in place at least 3 to 4 days. However, because the liquid graft described herein is composed of very small fragments of tissue, aggregates of cells and individual cells of fast healing buccal tissue, the process of engraftment will be significantly faster. Moreover, it may also be faster because the liquid graft is being injected into a fresh wound where wound healing processes are occurring. In addition, the liquid graft when applied consists of dissociated buccal mucosa tissue and a viscous carrier which binds it to the wound site. Thus, when using the liquid graft of the present invention, the mold (e.g., a catheter, stent, wound healing dressing, balloon) may need to be kept in place for as little as ¼, ½, 1, 2, 3, 6, 12, 18, 24, 36 or 48 hours, and is removed after ¼, ½, 1, 2, 3, 6, 12, 18, 24, 36 or 48 hours.

In accordance with this and all aspects of the disclosure, the liquid suspension of buccal mucosa tissue can be delivered and dispersed to a wound area using a syringe, a dual-barrel syringe and compatible catheter, angiocatheter, catheter, needle, spray device, a bulb and compatible catheter, bulb syringe, eye dropper, stent, brush, roller, or any combination of these instruments.

In one embodiment, a dual-barrel syringe is used to deliver and disperse the liquid suspension of buccal mucosa tissue. A dual-barrel syringe is particularly suitable for us if a two-part fibrin sealant (or an equivalent) is employed, with one component of the sealant in each barrel. The suspension of dissociated buccal mucosa tissue may be added to one or both of the barrels of the syringe, one or both of which may contain a port through which the suspension of dissociated buccal mucosa tissue can be added. A triple-barrel syringe may also be used, with one barrel used for each of the two fibrin sealant components (or if another dual component carrier is used, for each of those components), and the third barrel used for the suspension of dissociated buccal mucosa tissue; the three components can be mixed all at once; first one component of the fibrin sealant (or its equivalent) mixes with the suspension of dissociated buccal mucosa tissue and then both mix with the other component of the fibrin sealant (or its equivalent), or the two components of the fibrin sealant (or its equivalent) are mixed together and then the dissociated buccal mucosa tissue is mixed in. The mixing can occur prior to, during or immediately after dispersion of the suspension of dissociated buccal mucosa tissue onto the wound. A barrel of the syringe in which the suspension of buccal mucosa tissue is mixed with a component of the carrier may incorporate design features or elements that facilitate mixing of the buccal mucosa tissue with the component of the carrier, either within the barrel or while its contents are exiting through the syringe's tip, such as, for example, a plunger tip that spins as it is depressed (optionally having fins on its face), helical grooves or ribs in the tip or in the barrel, a bead incapable of blocking fluid flow out of the syringe (to facilitate mixing when the syringe is shaken, or a magnetic stirring rod (the syringe would be place on a magnetic mixer plate after the buccal mucosa tissue suspension was added)). If the components are not stored or provided in the syringe, the container holding either can include appropriate mixing enhancing mechanisms or design features.

Another aspect of the present disclosure is a suspension of dissociated buccal mucosa tissue that is used as a liquid graft for treatment of a wound or luminal wall defect. In accordance with this aspect of the disclosure, dissociated buccal mucosa tissue refers to buccal mucosa tissue that has been enzymatic and/or mechanically disrupted or dissociated from its natural state. When the liquid graft is dispersed over or otherwise delivered to the site of a wound, buccal mucosa tissue and cells are engrafted at the site of the wound, promoting healing.

In one embodiment, the dissociated buccal mucosa tissue in suspension comprises epithelial progenitor cells. In one embodiment, the suspended dissociated buccal mucosa tissue comprises fragments of stratified squamous epithelium and fragments of lamina propria. In one embodiment, the dissociated buccal mucosa tissue suspended in solution comprises epithelial progenitor cells and further comprises fragments of stratified squamous epithelium and/or fragments of lamina propria. In one embodiment, the dissociated buccal mucosa tissue suspended in solution comprises epithelial progenitor cells and fragments of lamina propria. In one embodiment, the dissociated buccal mucosa tissue is suspended in a solution that contains one or more components that increase the viscosity of the solution. In one embodiment, the dissociated buccal mucosa tissue is suspended in a viscous solution. In one embodiment, the dissociated buccal mucosa tissue is suspended in a solution that contains one or more growth factors, one or more antibiotics, one or more agents known to promote wound healing, or a combination thereof. In one embodiment, the liquid graft used for treatment of a wound or luminal wall defect in a patient is prepared using, and contains autologous buccal mucosa tissue.

The suspension of dissociated buccal mucosa tissue may be purified or enriched for a particular size of cells or fragments and/or for a particular cell type as described above. In one embodiment, the dissociated buccal mucosa tissue is enriched for epithelial progenitor cells to contain a higher concentration of epithelial progenitor cells than found in normal buccal mucosa tissue (about 20%, 30%, 40%, 50%, 60%, 70%, 80%, 90%, or >90% of the total cells in the suspended tissue).

Another aspect of the present disclosure is directed to kit for preparing a liquid buccal mucosal tissue graft. The kit comprises one or more instruments for dissociating buccal mucosa tissue, and one or more components suitable for suspension and/or delivery of the dissociated buccal mucosa tissue graft to a site on or in a subject that is in need of a liquid buccal mucosal tissue graft.

Instruments for mechanically dissociating the buccal tissue after biopsy include, without limitation, scalpel, cutting board or surface, sonicator, press mincer, chopper, strainer, grater, blades, mortar and pestle, tissue grinder, titrating syringes, pipettes, mincer, cell scraper, sieves, plastic or glass sterile cell culture tubes, and plates. The instruments for mechanically dissociating the tissue can be manual or powered, and may incorporate or include a container for collecting the dissociated tissue. The instruments may be attachments of another instrument not included in the kit, such as a container incorporating a chopper blade on a drive shaft that can be inserted into a separate motor unit.

The kit may optionally also contain reagents suitable for enzymatic dissociation of the buccal mucosa tissue. In one embodiment, the reagents are housed or packaged in a container into which biopsied buccal mucosa tissue can be placed and dissociated. Suitable enzymes for tissue dissociation are disclosed supra. The kit optionally also contains instruments for harvesting the buccal mucosa tissue. Suitable instruments for harvesting the buccal mucosa tissue include, without limitation, forceps, blade scalpel, punch biopsy (e.g., 4 mm-5 mm punch biopsy), fine sharp scissors, sterile gauze, sterile saline, and suture material.

The one or more components suitable for suspension and/or delivery of the dissociated buccal mucosa tissue graft may include solutions, or components of solutions suitable for washing, storing and/or delivering the buccal mucosa tissue graft as described supra. These solutions and/or components of solutions optionally include pharmaceutically acceptable saline solution (e.g., physiological or sterile saline solution), phosphate buffered saline (PBS), Ringer's serum, Ringer's lactate serum, autologous serum, and AB donor serum, and/or other excipients such as 4-(2-hydroxyethyl)-1-piperazine-ethanosulfonic acid (HEPES) buffer, bicarbonate buffer, heparins, platelet lysates, N-acetyl cysteine (NAC), and other nutrients for maintaining cell viability and integrity prior to dispersion at a wound site. These components may further comprise one or more growth factors, antibiotics, wound healing reagents, and/or reagents to increase the viscosity of the suspension (e.g., components of the fibrin sealant, hydrogel material, or medical honey) as described supra. Any of these components of the kit, i.e., growth factors, wound healing reagents, reagents to increase viscosity of the solution may be packaged together as mixtures of two or more components, or packaged separately to be combined at the time of use to produce a desired suspension or delivery solution when needed (i.e., at the time of buccal mucosa tissue harvest). Exemplary growth factors, antibiotics, wound healing reagents and reagents to increase the viscosity of the suspension (e.g., hydrogel materials) are described supra.

As described supra, the solutions for suspension and delivery of the buccal mucosa tissue may be the same or different and may include multiple solutions, each solution comprising one or more of the aforementioned components.

The kit optionally also contains one or more instruments suitable for delivery of the liquid buccal mucosal tissue graft. Instruments suitable for delivery of the liquid buccal mucosal tissue graft include, without limitation, syringes (e.g., dual or triple barrel syringes), catheters, angiocatheters, syringes and compatible catheters, bulbs and compatible catheters, bulb syringes, eye droppers, needles, spray devices, brushes, rollers, or stents, as described supra. Other instruments utilized in the delivery of the tissue graft include instruments such as, for example and without limitation, a stent, patch, balloon, catheter, etc. having a surface capable of holding the dissociated buccal mucosa tissue in place on or over the site of delivery (e.g., a wound site) or for accepting a viscous suspension of dissociated buccal mucosa tissue.

The kit optionally also contains an instrument for observing the process of dispersing the liquid suspension of buccal mucosa tissue to ensure that it is dispersed at the desired site (e.g., an endoscope). The kit optionally also contains an instrument or endoscopic attachment for making an incision or resection. The endoscope incorporating a cutting tool or to which a cutting tool has been attached is for use removing or incising scar tissue associated with a luminal stricture being treated in a subject.

The kit optionally also contains one or more containers for dissociating and housing harvested buccal mucosa tissue. Suitable containers include any standard cell culture plates, tubes, vials, bottles, beakers, flasks, or any other container capable of holding liquid. The kit may optionally also contain reagents and tools that facilitate the purification or enrichment of buccal mucosa cells and/or tissue as described supra.

The kit may contain any combination of the above described components. Each of the aforementioned solutions and reagents may be packaged as separate solutions or reagents, or two or more may be combined (for example, the solution for suspending and/or washing the dissociated buccal mucosa tissue may contain pharmaceutical grade saline and an antibiotic). The kit will also include packaging that will keep all its components together and preferably sterile, along with instructions describing the method of using the kit components to prepare suspension of dissociated buccal mucosa tissue for use as a liquid graft to treat a wound or luminal stricture.

In one embodiment, the kit contains an instrument for harvesting buccal mucosa tissue; an instrument for dissociating the buccal mucosa tissue; a solution for suspending the dissociated buccal mucosa tissue containing at least one antibiotic; a dual-barrel syringe; both components of a two component carrier (e.g., thrombin and fibrinogen complex); a rigid or flexible catheter or tube for dispersion of the dissociated buccal mucosa tissue at or into a wound site; and one of a catheter, stent, balloon or a patch to serve as a mold.

Another aspect of the present disclosure is directed to kit for preparing a liquid buccal mucosal tissue graft from disassociated buccal mucosa tissue or expanded cells from buccal mucosa tissue and delivering the liquid buccal mucosal tissue graft to an appropriate wound or surgical site. The kit comprises a viscous solution or components capable of increasing the viscosity of a solution and one or more instruments suitable for delivery of the liquid buccal mucosal tissue graft. The components capable of increasing the viscosity of a solution can be any of the components described above. Instruments for suitable delivery include any of the above described instruments.

The kit optionally comprises instruments for containing the liquid buccal mucosal tissue graft at the intended site, such as a catheter, stent, inflatable balloon or wound healing dressing. The kit optionally comprises any of the components suitable for suspension and/or delivery of the dissociated buccal mucosa tissue graft described above. The kit may contain any combination of the above described components, including but not limited to reagents, solutions, instruments, devices, packaging and instructions. The kit optionally comprises disassociated buccal mucosa tissue and/or expanded buccal mucosa cells. The kit optionally comprises a means or component which preserves the cells and/or tissue during transport (e.g., liquid nitrogen, dry ice, refrigeration unit).

The present invention is illustrated, but not limited, by the following examples.

### EXAMPLES

### Example 1 Liquid Buccal Mucosa Tissue Graft Implantation within a Defect in a Healthy Urethra

Creation of the Urethral Defect:

Four postpubertal male New Zealand white rabbits (Charles River, ref #052) ranging from 2.8 kg to 3.4 kg were used. Following the standard one-week acclimatization period, anesthesia was induced with 40 mg/kg ketamine hydrochloride and 6 mg/kg intramuscular xylazine then the genitalia were prepped with povidone-iodine solution. A 10-French pediatric urethrotome (Storz, Germany) was used to create a 1-cm longitudinal urethral mucosal incision ventrally at 6 o'clock position into the corpus spongiosum. The location of the defect was 2-3 cm proximal to the meatus and 1-2 cm distal to the membranous urethra. A 10-French silicone catheter was then placed into the bladder and the balloon inflated with 3 cc sterile water.

Buccal Mucosal Micro-Graft Preparation:

An 8-mm diameter buccal mucosa graft (“BMG”) was harvested with a circular punch biopsy (Miltex, York, Pa., ref # MTX-33-31AP). The mucosa was rinsed in a double antibiotic solution containing gentamicin 100 mg/L and vancomycin 1 g/L. The mucosa was defatted and mechanically minced it in 1 cc double antibiotic solution to create <1-mm fragments with sterile blades in a sterile metal dish. The minced mucosa was rinsed into a centrifuge tube and spun for 5 min at 3400 RPM to form a pellet. A carrier liquid for pellet re-suspension was prepared by diluting 1:1 the individual components of the surgical fibrin glue, TISSEEL® (Baxter, Deerfield, Ill.) with the double antibiotic solution. The tissue was then resuspended in two separate aliquots in 1-cc Eppendorf tubes, one containing a 150 microliters dilute fibrinogen analog and the other containing 150 microliters of dilute thrombin analog.

Buccal Micro-Graft Application:

To prevent solidification of the carrier glue within the syringe, each aliquot was injected separately into the urethra (thrombin-BMG solution first). A 14-Gauge angiocatheter fitted on a 1-cc syringe was placed through the meatus adjacent to the Foley catheter and the suspended micrograft solutions were injected. The Foley catheter was then secured in place with nonabsorbable monofilament suture and further protected with elastic wraps on their abdomens. Animals were fitted with neck cones until the catheters were discontinued one week postoperatively.

Specimen Collection:

The animals were sacrificed 3 weeks postoperatively by a lethal injection of pentobarbital 390 mg/10 pounds. The whole urethras and penises were harvested en-block immediately after euthanization. In detail, peno-scrotal skin webs were incised to release the penis, the penis was degloved and circumferentially dissected to the level of pubic ramus. The entire specimen was transected at the level of membranous urethra, suspended in 10% formaldehyde and sent for histological examination by a dedicated urologic pathologist.

Results:

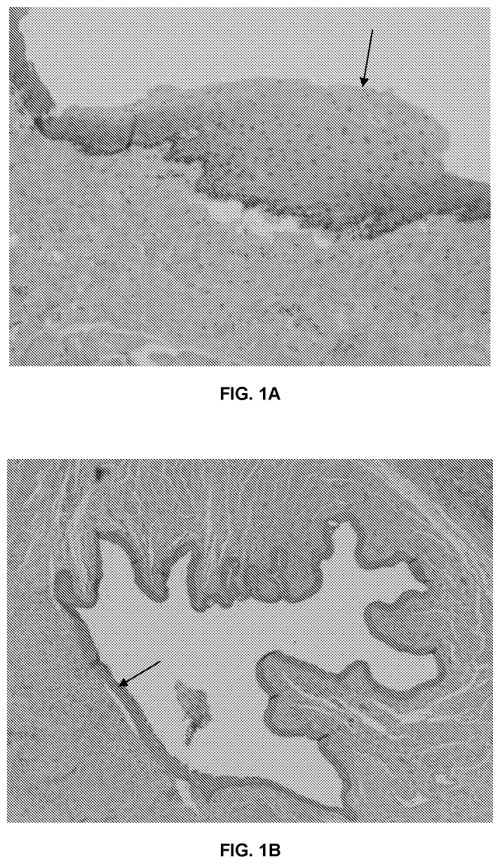

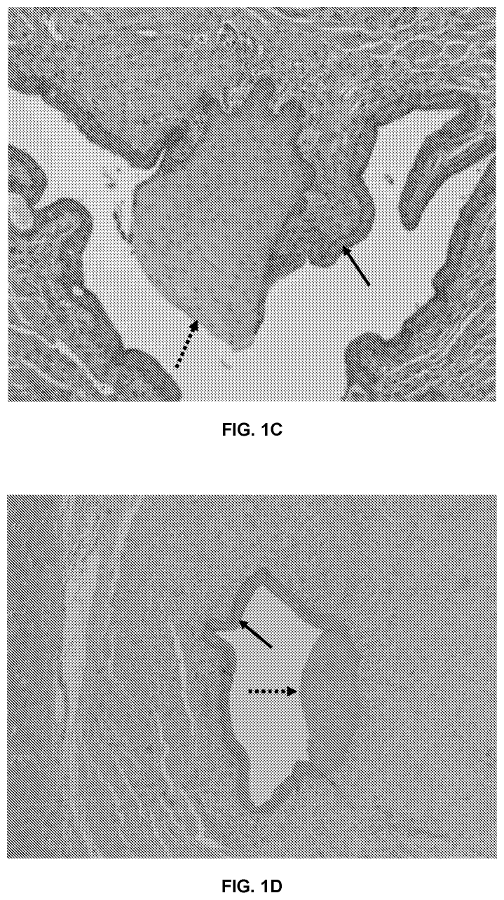

Of the 4 rabbits, 3 successfully underwent the procedure and one died during the induction of anesthesia before any surgical intervention. The urethral specimen of this last animal was used as a normal control. Two of the three treated animals showed histological evidence of buccal mucosal engraftment within the urethra. Representative micrographs of urethras from Phase I showing implantation of buccal mucosa can be seen in FIGS. 1A-1D. FIG. 1A is a photomicrograph of normal buccal mucosa taken from the cheek of a rabbit stained with hematoxylin and eosin (H+E staining). The arrow points at normal buccal mucosa epithelium (rabbit). FIG. 1B is a photomicrograph of a cross-section of normal untreated rabbit urethra with H&E staining. The arrow points at normal urethral lining. FIG. 1C shows a cross-section of rabbit urethra two weeks after liquid buccal mucosa administration (H&E staining). The dotted arrow points to the liquid buccal mucosa engrafted in the urethral defect in the rabbit. The solid arrow points at normal urethral epithelium. FIG. 1D shows a cross-section of rabbit urethra three weeks after liquid buccal mucosa administration (H&E staining). The dotted arrow points to liquid buccal mucosa engrafted in the urethral defect in the rabbit. The solid arrow points at normal urethral epithelium.

### Example 2—Liquid Buccal Mucosal Graft for Treatment of Urethral Stricture

### Materials and Methods for Example 2:

Induction of Stricture.

Twelve male New Zealand white rabbits (Charles River) between 2.8 to 3.6 kg were purchased and acclimatized for one week per protocol. Urethral strictures were induced in these rabbits following a modification of the electroresection protocol described by Faydaci et al., “Comparison of Two Experimental Models for Urethral Stricture in the Anterior Urethra of the Male Rabbit,” Urology 80(1):225.e7-11 (2012), which is hereby incorporated by reference in its entirety. Specifically, anesthesia was induced as described above. The abdomen was shaved for placement of a pediatric grounding pad (ConMed, ref #440-2400). Anesthesia was maintained with inhaled isoflurane. Prior to resection, a retrograde urethrogram (RUG) was performed under fluoroscopy using undiluted iohexol nonionic radiographic contrast (Omnipaque, GE Healthcare) to establish a baseline. A 10-French pediatric resectoscope (Storz, Germany) was used to create a 1 cm long mucosal defect in the urethra from 3 to 9 o'clock position starting 3 cm proximal to the meatus. Repeat RUG was done to confirm extravasation of contrast into corpus spongiosum. Animals were left without Foley catheters and were evaluated for signs of urinary retention. Four to six weeks later, rabbits were re-anesthetized and RUGs were again performed to assess development of a stricture. Animals that demonstrated radiographic evidence of stricture were randomly assigned in a 2:1 ratio to treatment vs control groups. Animals without evidence of stricture underwent one repeated electroresection protocol.

Treatment of Stricture.

Following the standard one-week acclimatization period, anesthesia was induced with 40 mg/kg ketamine hydrochloride and 6 mg/kg intramuscular xylazine then the genitalia were prepped with povidone-iodine solution. Anesthesia was maintained by inhaled isoflurane. Both groups underwent direct vision internal urethrotomy (DVIU) using 10-French pediatric urethrotome. Three to six ventral and lateral radial urethrotomy incisions were made to create at least a 10-French lumen adequate for passage of the scope. A 10-French silicone Foley catheter with a 3 cc balloon was placed into the urethra to drain the bladder after each urethrotomy. The treatment group underwent BMG harvest and preparation as described in Example 1. In brief, an 8-mm BMG fragment was washed in double antibiotic solution, mechanically minced in a sterile metal dish, centrifuged for 10 min and pellet divided in two 1-cc Eppendorf tubes. Each aliquot was suspended in 150 μL dilute components of fibrin glue. The components were not mixed to prevent early solidification of the liquid before the injection. Suspended cells from each tube were collected into individual 1-cc syringes fitted with 14-gauge angiocatheters and separately injected into urethra around the indwelling Foley catheter. The control animals did not have BMG harvest and received only pericatheter injection of dilute components of the fibrin glue without buccal mucosal fragments. In both groups catheters were secured at the meatus with non-absorbable nylon 3-0 sutures and further protected with elastic wraps on their abdomens. Animals were fitted with neck cones for 7 days until the removal of the catheters. The animals were euthanized at 8 weeks, 16 weeks, and 24 weeks post operatively having two experimental animals and one control animal at each time point. Prior to euthanization, final retrograde urethrograms were obtained for evidence of stricture recurrence.

Pathological Examination:

All specimens were fixed in 10% neutral buffered formalin for >6 hours, grossed taking serial sections, and embedded in paraffin wax according to standard protocol using a TissueTech™ processor overnight. The entire length of the urethra with attached corpora spongiosum and corpora cavernosum was grossed in 4 to 5 mm sections. Hematoxylin and eosin staining was performed on 5 μm tissue sections on glass slides. All slides were examined by light microscopy, with attention to the formation of a scar (composed of well-organized, dense collagen), acute inflammation, chronic inflammation, foreign body giant cell reaction, vascular density/granulation tissue formation, and engraftment of buccal mucosa within urethral cross sections. Histopathology images were captured using Nixon software on a Zeiss light microscope.

### Results:

Stricture Induction Results.

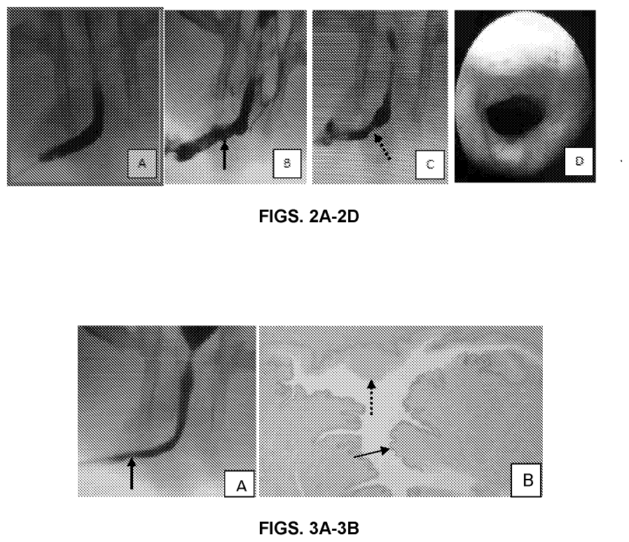

Of the 12 rabbits, 8 demonstrated radiological evidence of stricture 4-6 weeks after the first stricture induction attempt (FIGS. 2A-2D). FIG. 2A shows retrograde urethrogram before stricture induction. FIG. 2B is a retrograde urethrogram taken immediately after endoscopic stricture induction (epithelium resection) showing contrast extravasation into corpus spongiosum. The solid arrow points at the defect created to cause contrast extravasation. FIG. 2C is a retrograde urethrogram at 6 weeks after stricture induction showing evidence of urethral narrowing. The dotted arrow points at the resulting urethral stricture. FIG. 2D is an endoscopic view of strictured urethral segment. Additional two rabbits had strictures 8 weeks after the second induction attempt. The remaining two rabbits without strictures were euthanized and excluded from the study.

Treatment Outcomes.

A total of seven animals received treatment with liquefied BMG and 3 animals were controls. One of the treatment group animals demonstrated signs of weight loss and failure to thrive and was euthanized 2 weeks postoperatively. The urethra was harvested and had shown large area of BMG engraftment across the prior stricture segment (see FIGS. 3A and 3B). FIG. 3A is a retrograde urethrogram showing short area of strictured distal urethra (arrow) immediately before endoscopic DVIU and liquid graft injection, and FIG. 3B is a photomicrograph of the treated urethral segment 2 weeks post buccal mucosal tissue treatment (the dotted arrow indicates engrafted buccal mucosa, the solid arrow indicates normal urethral epithelium). A large area of buccal mucosa engraftment is seen covering approximately ⅓ circumference of the urethral lumen.

The remaining nine animals were thriving, and were euthanized at planned time intervals. FIGS. 4A-4C show stricture formation in a control animal that did not receive a liquid buccal mucosa tissue graft. FIG. 4A is a retrograde urethrogram showing stricture (arrow) before DVIU, and FIG. 4B is a retrograde urethrogram showing stricture (arrow) 8 weeks after DVIU without buccal mucosal graft. FIG. 4C is a photomicrograph of the H&E stained urethra at the level of stricture 8 weeks post DVIU showing extensive collagen deposition and fibrosis (arrows).

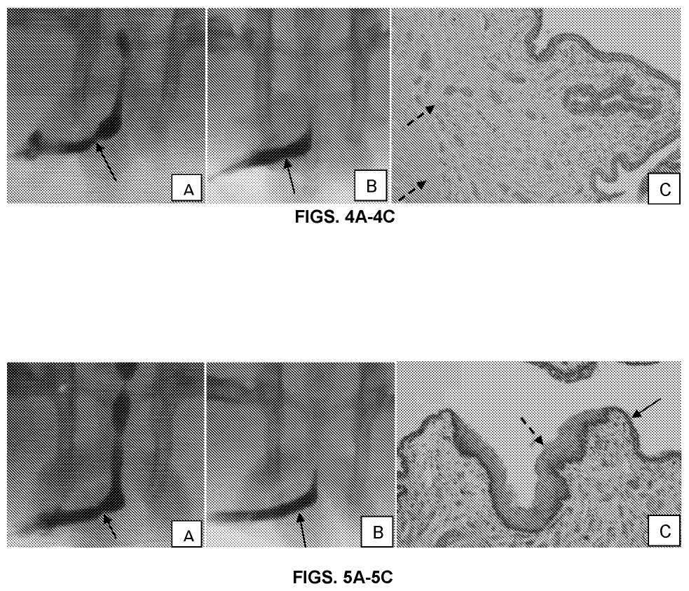

FIGS. 5A-5C show stricture formation and resolution in an animal treated with the liquid buccal mucosa tissue graft. FIG. 5A is a retrograde urethrogram showing stricture (arrow) before DVIU, and FIG. 5B is a retrograde urethrogram showing resolution of stricture (arrow) 8 weeks after DVIU with injection of liquid buccal mucosal graft. FIG. 5C is a photomicrograph of the H+E stained urethra at the level of stricture at 8 weeks after DVIU showing buccal mucosal engraftment within the urethra (dotted arrow). The solid arrow points to normal urethral lining.

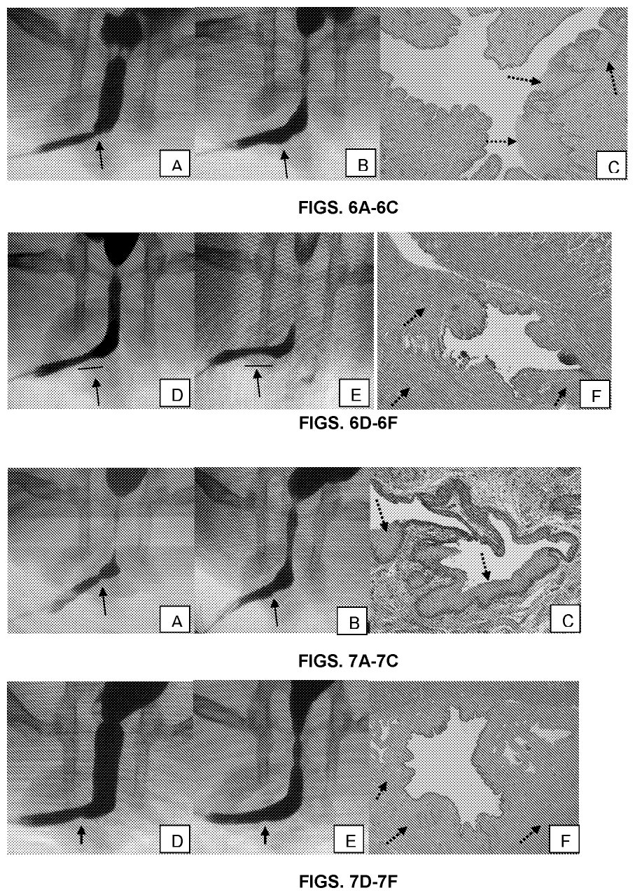

FIGS. 6A-6C show stricture formation and resolution in an animal treated with the liquid buccal mucosa tissue graft. FIG. 6A is a retrograde urethrogram showing stricture (arrow) before DVIU, and FIG. 6B is a retrograde urethrogram showing resolution of stricture (arrow) 16 weeks after DVIU with injection of liquid buccal mucosal graft. FIG. 6C is a photomicrograph of H+E staining of the urethra at the level of stricture at 16 weeks after DVIU showing buccal mucosal engraftment within the urethra (the dotted arrows indicate three areas of buccal mucosa engraftment). FIGS. 6D-6F show the control animal at 16 weeks. FIG. 6D is a retrograde urethrogram showing the stricture (arrow) before DVIU (solid line indicates length of stricture). FIG. 6E is a retrograde urethrogram showing the stricture (arrow) 16 weeks after treatment by DVIU without buccal mucosal graft (solid line indicates length of stricture). FIG. 6F shows H+E staining of the urethra at the level of stricture showing extensive collagen deposition and fibrosis (dotted arrows) at 20× magnification.

FIGS. 7A-7C show stricture formation and resolution in an animal treated with the liquid buccal mucosa tissue graft. FIG. 7A is a retrograde urethrogram showing stricture (arrow) before DVIU, and FIG. 7B is a retrograde urethrogram showing resolution of stricture (arrow) 24 weeks after DVIU with injection of liquid buccal mucosal graft. FIG. 7C is a photomicrograph of the urethra after H&E staining at the level of stricture at 24 weeks after DVIU showing buccal mucosal engraftment within the urethra (dotted arrows indicate areas of buccal mucosal engraftment within the urethra). FIGS. 7D-7F show the control animal at 24 weeks. FIG. 7D is a retrograde urethrogram showing the stricture (arrow) before DVIU (solid line indicates length of stricture). FIG. 7E is a retrograde urethrogram showing improved stricture (arrow) 24 weeks after treatment by DVIU without buccal mucosal graft. FIG. 7F shows H+E staining of the urethra at the level of stricture showing extensive collagen deposition and fibrosis (dotted arrows) at 20× magnification.

### Discussion of Example 2:

Urethroplasty, in its many forms, has been called a “gold standard” treatment for urethral stricture disease due to its high rate of success and long term durability (Barbagli et al., “Long-term Followup and Deterioration Rate of Anterior Substitution Urethroplasty,” I 192(3):808-13 (2014) and Chapple et al., “SIU/ICUD Consultation on Urethral Strictures: The Management of Anterior Urethral Stricture Disease Using Substitution Urethroplasty,” Urology 83(3 Suppl): S31-47 (2014), which are hereby incorporated by reference in their entirety. This is an underutilized treatment possibly due to unfamiliarity of most urologists with the techniques and the invasiveness of this type operation. Most urologists are more comfortable with the less technically challenging option of direct vision internal urethrotomy (DVIU) (Ferguson et al., “Minimally Invasive Methods for Bulbar Urethral Strictures: A Survey of Members of the AUA,” J Urol (2011); Anger et al., “Patterns of Management of Urethral Stricture Disease in the Veterans Affairs System,” Urology 78(2):454-458 (2011); Anger et al., “Trends in Stricture Management Among Male Medicare Beneficiaries: Underuse of Urethroplasty?,” J Urol 77(2):481-485 (2011); and Frank et al., “Urethroplasty: A Geographic Disparity in Care,” J Urol 187:2124-2127 (2012), which are hereby incorporated by reference in their entirety). This endoscopic operation, albeit minimally invasive, has been shown to be minimally effective with cure rates as low as 8-12% (Santucci et al., “Urethrotomy Has a Much Lower Success Rate Than Previously Reported,” J Urol 183:1859-6 (2010) and Al Taweel et al., “Visual Internal Urethrotomy for Adult Male Urethral Stricture Has Poor Long-Term Results,” Adv Urol. 2015:656459 doi:10.1155/2015/656459 (2015), which are hereby incorporated by reference in their entirety). Several groups have proposed methods to improve success of endoscopic internal urethrotomy by inhibiting fibroblast proliferation with Mitomycin C, triamcinolone or captopril gel (Mazdak et al., “Effect of Mitomycin C on Anterior Urethral Stricture Recurrence After Internal Urethrotomy,” Eur Urol. 51(4):1089-92 (2007); Mazdak et al., “Internal Urethrotomy and Intraurethral Submucosal Injection of Triamcinolone in Short Bulbar Urethral Strictures,” Int Urol Nephrol. 42(3):565-8 (2010); Tavakkoli et al., “Triamcinolone Injection Following Internal Urethrotomy for Treatment of Urethral Stricture,” Urol J. 8(2):132-6 (2011); Modh et al., “Outcomes of Direct Vision Internal Urethrotomy for Bulbar Urethral Strictures: Technique Modification with High Dose Triamcinolone Injection,” Adv Urol. 2015:281969 (2015); and Shirazi et al., “Effect of Intraurethral Captopril Gel on the Recurrence of Urethral Stricture After Direct Vision Internal Urethrotomy: Phase II Clinical Trial,” Int J Urol. 14(3):203-8 (2007), which are hereby incorporated by reference in their entirety). Mazdak et al randomized 40 patients to receive DVIU with or without intraurethral mitomycin-C injection (Mazdak et al., “Effect of Mitomycin C on Anterior Urethral Stricture Recurrence After Internal Urethrotomy,” Eur Urol. 51(4):1089-92 (2007), which is hereby incorporated by reference in its entirety). After a short term follow up of 6 months, strictures recurred in 2/20 (10%) treated patients compared to 10/20 (50%) control patients. The same group randomized 50 patients to DVIU with or without triamcinolone injection and showed significant short term reduction of stricture recurrence rate in the treated group (Mazdak et al., “Internal Urethrotomy and Intraurethral Submucosal Injection of Triamcinolone in Short Bulbar Urethral Strictures,” Int Urol Nephrol. 42(3):565-8 (2010), which is hereby incorporated by reference in its entirety). Other trials have not shown consistent benefits of fibroblast inhibiting agents in treatment of urethral strictures or bladder neck contractures (Tavakkoli et al., “Triamcinolone Injection Following Internal Urethrotomy for Treatment of Urethral Stricture,” Urol J. 8(2):132-6 (2011) and Redshaw et al., “Intralesional Injection of Mitomycin C at Transurethral Incision of Bladder Neck Contracture May Offer Limited Benefit: TURNS Study Group,” J Urol. 193(2):587-92 (2015), which are hereby incorporated by reference in their entirety). The general theory behind these methods is that if the scar formation is delayed, the normal epithelization of the urethrotomy defect will stabilize the urethral lumen and keep it open.

The theory behind the method described in the studies herein relies on expedited epithelization of the wound with buccal mucosal cells which are thought to exhibit “preferential” wound healing properties (Ishii et al., “Expression of p75(NGFR), a Proliferative and Basal Cell Marker, in the Buccal Mucosa Epithelium during Re-epithelialization,” Acta Histochem Cytochem. 47(4):145-53 (2014); Board-Davies et al., “Oral Mucosal Lamina Propria-Progenitor Cells Exert Antibacterial Properties via the Secretion of Osteoprotegerin and Haptoglobin Stem Cells,” Transl Med. 4(11):1283-1293 (2015); Davies et al., “A Multipotent Neural Crest-derived Progenitor Cell Population is Resident Within the Oral Mucosa Lamina Propria,” Stem Cells Dev. 19(6):819-30 (2010); Turabelidze et al., “Intrinsic Differences Between Oral and Skin Keratinocytes,” PLoS One 9(9):e101480 (2014); and Kuroki et al., “Epithelialization in Oral Mucous Wound Healing in Terms of Energy Metabolism,” Kobe J Med Sci. 55(1):E5-E15 (2009), which are hereby incorporated by reference in their entirety). It has been noted that buccal mucosal cells have increased metabolism (Kuroki et al., “Epithelialization in Oral Mucous Wound Healing in Terms of Energy Metabolism,” Kobe J Med Sci. 55(1):E5-E15 (2009), which is hereby incorporated by reference in its entirety) and contain numerous fibroblast and epithelial progenitor cells with stem cell properties (Ishii et al., “Expression of p75(NGFR), a Proliferative and Basal Cell Marker, in the Buccal Mucosa Epithelium during Re-epithelialization,” Acta Histochem Cytochem. 47(4):145-53 (2014) and Kuroki et al., “Epithelialization in Oral Mucous Wound Healing in Terms of Energy Metabolism,” Kobe J Med Sci. 55(1):E5-E15 (2009), which are hereby incorporated by reference in their entirety). Buccal sheet grafts have been extensively used in urethral reconstruction for several decades and their restorative properties have attracted the attention of regenerative scientists in numerous other specialties including, cornea reconstruction, esophageal stricture treatment, and, skin burn treatment (Lida et al., “Development of a Tissue-engineered Human Oral Mucosa Equivalent Based on an a Cellular Allogeneic Dermal Matrix: A Preliminary Report of Clinical Application to Burn Wounds,” Scand J Plast Reconstr Surg Hand Surg. 39(3):138-46 (2005); Bardag-Gorce et al., “Carrier-free Cultured Autologous Oral Mucosa Epithelial Cell Sheet (CAOMECS) for Corneal Epithelium Reconstruction: A Histological Study,” Ocul Surf. 13(2):150-63 (2015); and Kobayashi et al., “Prevention of Esophageal Strictures After Endoscopic Submucosal Dissection,” World J Gastroenterol. 20(41):15098-109 (2014), which are hereby incorporated by reference in their entirety). However, the utilization of buccal sheet grafts for the treatment of urethral strictures has been limited given the invasiveness of reconstructive surgery and availability of alternative, yet less effective, treatment options.

Endoscopic attempts on treatment of urethral stricture were proposed by Naudé J H., “Endoscopic Skin-Graft Urethroplasty,” World J Urol. 16(3):171-4 (1998), which is hereby incorporated by reference in its entirety, and involve urethrotomy augmented by a skin graft. This endoscopic method relied on placement of sutures through the perineum into the urethrotomy, endoscopic externalization of the sutures through the meatus, securing the graft and delivering (parachuting) the graft into the wound. This intricate method was used in 53 patients with success rates of 95% at 2 years, but is technically challenging and further relied on specialized catheter to keep the graft in place. Several other variations of this method exist and involve either an open perineal counter-incision for proximal graft fixation, a suprapubic access, or a specialized catheter for delivery and fixation of the graft (Kuyumcuoglu et al., “Antegrade Endourethroplasty With Free Skin Graft for Recurrent Vesicourethral Anastomotic Strictures After Radical Prostatectomy,” J Endourol. 24(1):63-7 (2010); Gaur D D., “Endourethral Urethroplasty—Use of a New Catheter,” J Urol. 130(5):905-8 (1983); and Seth et al., “Hybrid Minimally Invasive Urethroplasty for Pan-anterior Urethral Strictures: Initial Results,” Urol Int. 89(1):116-9 (2012), which are hereby incorporated by reference in their entirety). Neither of these methods have achieved widespread use due to the need of specialized instruments and/or due to being technically challenging (Naudé et al., “What is the Place of Internal Urethrotomy in the Treatment of Urethral Stricture Disease?,” Nat Clin Pract Urol. 2(11):538-45 (2005), which is hereby incorporated by reference in its entirety).

The method described herein relies on pericatheter injection of random BM micro-grafts dispersed in a readily available surgical sealant. This simplifies both the delivery and fixation of the graft avoiding specialized instruments, advanced endoscopy skills, and the need for a counter incision. The proposed method has several advantages, including, but not limited to: 1) it provides a simple method for stricture repair by any urologist familiar with direct vision internal urethrotomy, 2) it allows for immediate use of autologous buccal mucosal cells not requiring chemical dissociation or expansion in external facility, and 3) it involves minimal preparation of the graft with instruments and materials readily available in any operative room. The use of commercially available surgical fibrin glue could be easily substituted for autologous fibrin preparation.

While the studies described herein involved the use of a minimal number of animal, the goals of this study were clearly achieved. First, the injected minced buccal mucosal micrografts show engraftment within created urethral defect in a healthy rabbit model. Second, buccal mucosal micrografts implant within the DVIU defect in a strictured urethra in a rabbit model and demonstrate no radiographic stricture recurrence at 6 months follow up.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of treating a wound in a subject, said method comprising:
providing a liquid suspension of dissociated buccal mucosa tissue and
dispersing the liquid suspension of dissociated buccal mucosa tissue over the wound thereby treating the wound in said subject.

2. The method of claim 1, wherein said wound is located within a urogenital tract duct or organ, a gastrointestinal tract duct or organ, or another lumen containing duct or organ of said subject.

3. The method of claim 2, wherein said wound is caused by removal or incision of scar tissue within said urogenital tract duct or organ, within said gastrointestinal tract duct or organ, or other lumen containing duct or organ of said subject.

4. The method of claim 2 further comprising:
positioning a device selected from a group consisting of a catheter, a stent, an inflatable balloon, and a wound healing dressing, within the duct or organ adjacent the wound prior to said dispersing, wherein said liquid suspension of dissociated buccal mucosa tissue is dispersed around the device over the wound.

5. The method of claim 1, wherein said liquid suspension of dissociated buccal mucosa tissue comprises at least one of epithelial progenitor cells from buccal mucosa tissue, other cells from buccal mucosa tissue, aggregates of cells from buccal mucosa tissue, and fragments of buccal mucosa tissue.

6. The method of claim 1, wherein said liquid suspension of dissociated buccal mucosa tissue is viscous or comprises one or more components that increase the viscosity of said suspension either prior to, concurrent with, or after said dispersing.

7. The method of claim 1, wherein said providing comprises:
harvesting buccal mucosa tissue from said subject;
dissociating the harvested buccal mucosa tissue; and
suspending the dissociated buccal mucosa in a carrier solution to form said liquid suspension of dissociated buccal mucosa tissue suitable for said dispersing.

8. The method of claim 1, wherein said liquid suspension of dissociated buccal mucosa tissue further comprises one or more growth factors, a solution comprising one or more wound-healing factors, a cell nutrient solution, physiological saline solution, an antibiotic solution, or combinations thereof.

9. A kit for preparing a liquid buccal mucosal tissue graft, said kit comprising:
one or more instruments and/or reagents for dissociating buccal mucosa tissue, and
one or more components suitable for suspension of the dissociated buccal mucosa tissue.

10. The kit of claim 9 further comprising:
one or more instruments suitable for delivery of the suspension of dissociated buccal mucosa tissue.

11. The kit of claim 9, wherein the one or more instruments and/or reagents for dissociating buccal mucosa tissue is selected from the group consisting of an enzyme, a chopper, a sieve, a mortal and pestle, a scalpel and cutting surface, a press mincer, and a tissue grinder.

12. The kit of claim 9, further comprising:
instruments for harvesting buccal mucosa tissue and
one or more containers for housing harvested buccal mucosa tissue.

13. A kit for delivery of disassociated buccal mucosa tissue as a liquid buccal mucosa tissue graft, said kit comprising:
a solution suitable for suspending dissociated buccal mucosa tissue, wherein said solution is a viscous solution or contains one or more components capable of increasing the viscosity of the solution, and
one or more instruments suitable for delivery of the solution containing the dissociated buccal mucosa tissue.

14. The kit of claim 9 further comprising:
one or more instruments selected from a catheter, a stent, an inflatable balloon, a wound healing dressing, an endoscopy device, endoscopy device incorporating a cutting tool, chopper, sieve, mortar and pestle, scalpel, cutting board or surface, press mincer, tissue grinder, single-barrel syringe, double-barrel syringe, triple barrel syringe and combinations thereof.

15. The kit of claim 13, further comprising dissociated buccal mucosa tissue.

16. The kit of claim 9, further comprising one or more components selected from the group consisting of a solution comprising one or more growth factors, a solution comprising one or more wound-healing factors, a cell nutrient solution, physiological saline solution, an antibiotic solution, one or more components capable of increasing the viscosity of a solution, a viscous solution, and combinations thereof.

17. A liquid graft comprising dissociated buccal mucosa tissue suspended in a solution.

18. The liquid graft of claim 17, wherein said dissociated buccal mucosa tissue comprises at least one of epithelial progenitor cells from buccal mucosa tissue, other cells from buccal mucosa tissue, aggregates of cells from buccal mucosa tissue, fragments of stratified squamous epithelium from buccal mucosa tissue, and/or fragments of lamina propria from buccal mucosa tissue.

19. The liquid graft of claim 17, wherein the solution is viscous and/or contains one or more components capable of increasing the viscosity of the solution.
